(19) United States
(12) Patent Application Publication
(10) Pub. No.: US 2025/0268610 A1
(43) Pub. Date: Aug. 28, 2025

(54) DRILL GUIDE SYSTEM FOR INTEROSSEOUS LIGAMENT REPAIR

(71) Applicant: Skeletal Dynamics, Inc., Miami, FL (US)

(72) Inventors: Jorge L. Orbay, Miami, FL (US); Daniel A. Sixto, Miami, FL (US); Gabriel Mesa, Miami, FL (US); Nicolas E. Zedan, Pembroke Pines, FL (US); Roger Pisarnwongs, Santa Clarita, CA (US); Tom Weisel, Ventura, CA (US)

(21) Appl. No.: 19/051,896

(22) Filed: Feb. 12, 2025

Related U.S. Application Data
(60) Provisional application No. 63/556,571, filed on Feb. 22, 2024.

(51) Int. Cl. A61B 17/17; A61B 17/00; A61B 17/88

(52) U.S. Cl. CPC A61B 17/1714; A61B 17/8897; A61B 2017/00407

(57) ABSTRACT

A guide and method for drilling a bone tunnel at a drill angle comprising a guide arm having an elongated segment joined to a connecting segment, a guide body having a hook terminating in a hook tip adapted to engage a distal side of the bone, a k-wire guide, and a locking rod; the elongated segment comprising a k-wire guide tunnel adapted to receive the k-wire guide; the connecting segment comprising a distal end having a guide arm tunnel; a longitudinal axis of the guide arm tunnel and a longitudinal axis of the k-wire guide tunnel converging at an angle equal to the drill angle; the guide body adapted to receive the connecting segment distal end and form a locking rod tunnel adapted to receive the locking rod; the k-wire guide and the locking rod each having a tip end adapted to engage a proximal side of the bone.

### CLAIM OF PRIORITY

This application is being filed as a non-provisional patent application under 35 U.S.C. § 111(a) and 37 CFR § 1.53(b). This application claims priority under 35 U.S.C. § 119(e) to U.S. provisional patent application Ser. No. 63/556,571 filed on Feb. 22, 2024, the contents of which are incorporated herein by reference.

### TECHNICAL FIELD

The technical field relates generally to surgical devices and systems and, in particular, to a drill guide system for drilling an angled tunnel into a bone for use in bone-to-bone fixation for ligament or tendon reconstruction.

### BACKGROUND

An interosseous membrane is a connective tissue that joins two bones and acts as a ligament. In the forearm, for example, the interosseous membrane connects the radius and ulna and assists in providing longitudinal stability in the forearm. Rupture of the interosseous membrane can result in instability and impact the rotation of the joints. However, interosseous membrane injuries can be difficult to treat.

During bone-to-bone fixation for ligament or tendon reconstruction, surgical drilling is used to create a tunnel through each of the two bones. In order to yield the correct tension to provide for the best anatomical response, each of the corresponding bone tunnels must be drilled at optimal and precise angles to create alignment between the bone tunnels. However, due to the proximity and complexity of the bones, it can be difficult to precisely align the bone tunnels to achieve the best anatomical response.

Therefore, there exists a need for a drilling guide that provides for precise alignment of the bone tunnels.

### SUMMARY

It is among the objects of this system to overcome the limitations of the heretofore-known devices by providing inventive features to achieve: a.) optimal positioning of a bone tunnel to yield the correct anatomical response; b.) precise alignment of corresponding bone tunnels; and c.) interchangeable instrumentation allowing for an efficient surgical procedure.

It is an object of the present system to provide a guide for drilling a tunnel through a bone at a drill angle. In one embodiment, the guide comprises a guide arm comprising an elongated segment joined to a connecting segment; a guide body comprising a first section and a second section; a k-wire guide; and a locking rod.

The elongated segment of the guide arm comprises a k-wire guide tunnel having a longitudinal axis, an entry, and an exit. The connecting segment comprises a distal end with a guide arm tunnel having a longitudinal axis. The guide arm tunnel further comprises an entry and an exit. In some embodiments, the longitudinal axis of the guide arm tunnel and the longitudinal axis of the k-wire guide tunnel converge at an angle equal to the drill angle. In further embodiments, the connecting segment may optionally be arcuate describing an arc equal to the drill angle. In embodiments having an arcuate connecting segment, the guide arm tunnel of the distal end is oriented substantially normal to the arc.

The first section of the guide body comprises an opening adapted to receive the distal end of the guide arm connecting segment. The first section further comprises an upper locking rod hole and a lower locking rod hole that are adapted to align with the guide arm tunnel to form a locking rod tunnel. The second section of the guide body comprises a hook optionally terminating in a hook tip. The hook tip is adapted to engage a distal side of the bone.

The k-wire guide is adapted to be received by the k-wire guide tunnel of the elongated segment of the guide arm. The k-wire guide comprises a proximal end, a distal end, and a shaft. The shaft further comprises a k-wire tunnel bored through the shaft. The k-wire tunnel is adapted to receive a k-wire. The proximal end of the k-wire guide may optionally further comprise a push pad. The distal end of the k-wire guide further comprises a tip end adapted to engage a proximal side of the bone.

The shaft of the k-wire guide optionally further comprises a plurality of ratchet teeth adapted for locking and unlocking the k-wire guide at a desired position. In at least some embodiments, the plurality of ratchet teeth on the shaft of the k-wire guide may optionally be radially oriented or straight horizontal cuts. The guide arm further comprises a k-wire guide ratchet mechanism adapted to engage the ratchet teeth on the k-wire guide. The k-wire guide ratchet mechanism is further adapted to permit the k-wire guide to slide freely in a direction from the entry to the exit of the k-wire guide tunnel, and to prevent the k-wire guide from sliding in a direction from the exit to the entry of the k-wire guide tunnel.

In a further embodiment, the guide may further comprise a k-wire guide button to disengage the k-wire guide ratchet mechanism from the ratchet teeth on the k-wire guide and permit the k-wire guide to slide in the direction from the exit to the entry of the k-wire guide tunnel.

The locking rod is adapted to be received by the locking rod tunnel that is formed by the upper and lower locking rod holes aligning with the guide arm tunnel. The locking rod comprises a proximal end, a distal end, and a shaft. The proximal end of the locking rod may optionally further comprise a push pad. The distal end of the locking rod further comprises a tip end adapted to engage the proximal side of the bone.

The shaft of the locking rod further comprises a plurality of ratchet teeth adapted for locking and unlocking the locking rod at a desired position. In at least some embodiments, the plurality of ratchet teeth on the shaft of the locking rod may optionally be radially oriented or straight horizontal cuts. The guide arm further comprises a locking rod ratchet mechanism adapted to engage the ratchet teeth on the locking rod. The locking rod ratchet mechanism is further adapted to permit the locking rod to slide freely in a direction from the upper to the lower locking rod hole, and to prevent the locking rod from sliding in a direction from the lower to the upper locking rod hole.

In a further embodiment, the guide may further comprise a locking rod button to disengage the locking rod ratchet mechanism from the ratchet teeth on the locking rod and permit the locking rod to slide in the direction from the lower to the upper locking rod hole.

In at least one alternate embodiment, the drill angle of the guide is 45 degrees.

In at least one further embodiment, the shaft of the locking rod may further comprise a channel at the distal end of the locking rod, and the first section of the guide body may further comprise a pin adapted to intersect with the locking rod tunnel. The pin of the guide body may be further adapted to engage the channel on the shaft of the locking rod to secure and prevent rotation of the locking rod when the locking rod is received by the locking rod tunnel.

In at least one other embodiment, the shaft of the k-wire guide may optionally further comprise a channel at the distal end of the k-wire guide, and the elongated segment of the guide arm may optionally further comprise a pin adapted to intersect with the k-wire guide tunnel. The pin of the guide arm may be further adapted to engage the channel on the shaft of the k-wire guide to secure and prevent rotation of the k-wire guide when the k-wire guide is received by the k-wire guide tunnel.

In one further embodiment, the k-wire guide may optionally further comprise a distal segment and a proximal segment. The distal segment is located at the distal end of the k-wire guide. The distal segment comprises a distal end, a proximal end, and a shaft therebetween with a distal segment tunnel bored through the shaft. In such an embodiment, the distal end of the distal segment comprises the tip end of the k-wire guide, and the shaft of the distal segment optionally further comprises the channel adapted to be engaged by the optional pin of the elongated segment of the guide arm. The proximal segment is located at the proximal end of the k-wire guide. The proximal segment comprises a distal end, a proximal end, and a shaft therebetween with a proximal segment tunnel bored through the shaft. In such an embodiment, the proximal end of the proximal segment may comprise the optional push pad of the k-wire guide, and the shaft of the proximal segment further comprises the plurality of ratchet teeth of the k-wire guide. The proximal end of the distal segment and the distal end of the proximal segment are adapted to removably attach to form the k-wire guide and align the distal segment tunnel with the proximal segment tunnel to form the k-wire tunnel. In at least some further embodiments, the distal end of the k-wire guide proximal segment may optionally further comprise an internal thread adapted to rotationally engage an external thread of the proximal end of the k-wire guide distal segment. In further embodiments, the optional channel of the k-wire guide and the optional pin of the elongated segment are adapted to prevent rotation of the k-wire guide distal segment while allowing linear translation of the k-wire guide distal segment and rotation of the k-wire guide proximal segment.

In at least one further embodiment, the locking rod may optionally further comprise a distal segment and a proximal segment. The distal segment is located at the distal end of the locking rod. The distal segment comprises a distal end, a proximal end, and a shaft therebetween. In such an embodiment, the distal end of the distal segment comprises the tip end of the locking rod, and the shaft of the distal segment may optionally further comprise the channel adapted to be engaged by the optional pin of the first section of the guide body. The proximal segment is located at the proximal end of the locking rod. The proximal segment comprises a distal end, a proximal end, and a shaft therebetween. In such an embodiment, the proximal end of the proximal segment may comprise the optional push pad of the locking rod, and the shaft of the proximal segment further comprises the plurality of ratchet teeth of the locking rod. The proximal end of the distal segment and the distal end of the proximal segment are adapted to removably attach to form the locking rod. In at least some further embodiments, the distal end of the locking rod proximal segment optionally further comprises an internal thread adapted to rotationally engage an external thread of the proximal end of the locking rod distal segment. In further embodiments, the optional channel of the locking rod and the optional pin of the first section of the guide body are adapted to prevent rotation of the locking rod distal segment while allowing linear translation of the locking rod distal segment and rotation of the locking rod proximal segment.

It is a further object to provide a method of using a guide to facilitate drilling a tunnel through a bone at a drill angle. In at least one embodiment, the method comprises placing a hook of the guide at a desired insertion point on a distal side of the bone. The guide may further comprise a k-wire guide tunnel and a locking rod tunnel. In at least one embodiment, a hook tip of the hook may engage the distal side of the bone when placing the hook of the guide at the desired insertion point on the distal side of the bone.

Next, a locking rod may be inserted into the locking rod tunnel until the locking rod contacts a proximal side of the bone. In some embodiments, one of a plurality of ratchet teeth on a shaft of the locking rod may be engaged by a locking rod ratchet mechanism on the guide. In some further embodiments, a tip end of the locking rod may engage the proximal side of the bone when inserting the locking rod into the locking rod tunnel until the locking rod contacts the proximal side of the bone.

Thereafter, a k-wire guide may be inserted into the k-wire guide tunnel until the k-wire guide contacts the proximal side of the bone such that the k-wire guide forms an angle with the locking rod equal to the drill angle. The k-wire guide may comprise a k-wire tunnel. In some embodiments, one of a plurality of ratchet teeth on a shaft of the k-wire guide may be engaged by a k-wire guide ratchet mechanism on the guide. In some further embodiments, a tip end of the k-wire guide may engage the proximal side of the bone when inserting the k-wire guide into the k-wire guide until the k-wire guide contacts the proximal side of the bone.

Next a k-wire may be inserted into the k-wire tunnel of the k-wire guide and drilled through the proximal side of the bone to the desired insertion point on the distal side of the bone.

Thereafter, the k-wire guide may be removed from the k-wire guide tunnel of the guide and the locking rod may be removed from the locking rod tunnel of the guide. In embodiments where the k-wire guide has a tip end that engaged a proximal side of the bone, the tip end of the k-wire guide may be disengaged prior to removing the k-wire guide from the k-wire tunnel. In embodiments where one of a plurality of ratchet teeth of the k-wire guide was engaged by a k-wire guide ratchet mechanism, the k-wire guide may be disengaged from the guide by pushing on a k-wire guide button on the guide to release the k-wire guide ratchet mechanism prior to removing the k-wire guide from the k-wire guide tunnel. Similarly, in embodiments where the locking rod has a tip end that engaged a proximal side of the bone, the tip end of the locking rod may be disengaged prior to removing the locking rod from the locking rod tunnel. In embodiments where one of a plurality of ratchet teeth of the locking rod was engaged by a locking rod ratchet mechanism, the locking rod may be disengaged from the guide by pushing on a locking rod button on the guide to release the locking rod ratchet mechanism prior to removing the locking rod from the locking rod tunnel.

The guide may then be removed from the bone. In embodiments where a hook tip of the hook engaged the distal side of the bone, the hook tip may be disengaged prior to removing the guide from the bone.

Next, a drill may be aligned over the k-wire that is inserted in the bone and the drill may then be drilled from the proximal side to the distal side of the bone to form a bone tunnel. The bone tunnel extends from the proximal side to distal side of the bone.

Thereafter, the drill and the k-wire may be removed from the bone. In at least some embodiments, the method may further comprise repeating the aforementioned steps at an appropriate position on a second corresponding bone to form a second corresponding bone tunnel at the drill angle.

Although the system and method are illustrated and described herein as embodied for use in interosseous ligament repair, it is nevertheless not intended to be limited to only the details shown, since various modifications and structural changes may be made therein without departing from the spirit of the invention and within the scope and range of equivalents of the claims.

The construction of the invention, however, together with additional objects and advantages thereof will be best understood from the following description of the specific disclosed embodiments when read in connection with the accompanying drawings.

### BRIEF DESCRIPTION OF THE DRAWINGS

FIG. 1 illustrates an isometric view of a guide for drilling a tunnel through a bone at a drill angle in accordance with one embodiment.

FIG. 2 illustrates an exploded view of the guide of FIG. 1 in accordance with one embodiment.

FIGS. 3 and 4 illustrate isometric views of the guide engaged with a bone in accordance with one embodiment.

FIG. 5 illustrates a top view of a guide arm in accordance with one embodiment.

FIG. 6 illustrates a cross-sectional view of the guide arm of FIG. 5 showing additional features in accordance with one embodiment.

FIGS. 7 and 8 illustrate isometric views of the guide arm of FIG. 5 showing additional features in accordance with one embodiment.

FIG. 9 illustrates a side view of a guide body in accordance with one embodiment.

FIGS. 10 and 11 illustrate isometric views of the guide body of FIG. 9 showing additional features in accordance with one embodiment.

FIG. 12 illustrates a cross-sectional view of the guide body of FIG. 9 showing additional features in accordance with one embodiment.

FIGS. 13 and 14 illustrate isometric views of the guide arm received by the guide body in accordance with one embodiment.

FIG. 15 illustrates a cross-sectional view of the guide arm received by the guide body of FIGS. 13 and 14 showing additional features in accordance with one embodiment.

FIGS. 16 and 17 illustrate isometric views of a k-wire guide in accordance with one embodiment.

FIG. 18 illustrates a cross-sectional view of the k-wire guide of FIGS. 16 and 17 showing additional features in accordance with one embodiment.

FIGS. 19 and 20 illustrate isometric views of a locking rod in accordance with one embodiment.

FIG. 21 illustrates a cross-sectional view of the locking rod of FIGS. 19 and 20 showing additional features in accordance with one embodiment.

FIGS. 22, 23 and 24 illustrate isometric views of a suture tensioner in accordance with another embodiment.

FIG. 25 illustrates an exploded view of the suture tensioner of FIGS. 22, 23 and 24 in accordance with one embodiment.

FIG. 26 illustrates a bottom view of a guide arm received by a guide body, wherein the guide arm has a pin engaging a channel of a k-wire guide and the guide body has a pin engaging a channel of a locking rod in accordance with one embodiment, wherein the guide arm, guide body, k-wire guide and locking rod are shown, for illustrative purposes only, as transparent to show additional features.

FIG. 27 illustrates an enlarged view of FIG. 26 showing the guide arm with the pin and having received the k-wire guide in accordance with one embodiment, wherein the guide arm and the k-wire guide are shown, for illustrative purposes only, as transparent to show additional features.

FIG. 28 illustrates an enlarged view of FIG. 26 of the guide body with the pin and having received the locking rod in accordance with one embodiment, wherein the guide body and the locking rod are shown, for illustrative purposes only, as transparent to show additional features.

FIG. 29 illustrates an isometric view of a k-wire guide with a distal segment and a proximal segment attached in accordance with another embodiment.

FIG. 30 illustrates an exploded isometric view of the k-wire guide of FIG. 29 showing additional features in accordance with one embodiment.

FIG. 31 illustrates a cross-sectional view of the k-wire guide of FIG. 29 with the distal segment and the proximal segment attached showing additional features in accordance with one embodiment.

FIG. 32 illustrates an isometric view of a locking rod with a distal segment and a proximal segment attached in accordance with another embodiment.

FIG. 33 illustrates an exploded isometric view of the locking rod of FIG. 32 showing additional features in accordance with one embodiment.

FIG. 34 illustrates a cross-sectional view of the locking rod of FIG. 32 with the distal segment and the proximal segment attached showing additional features in accordance with one embodiment.

### DETAILED DESCRIPTION

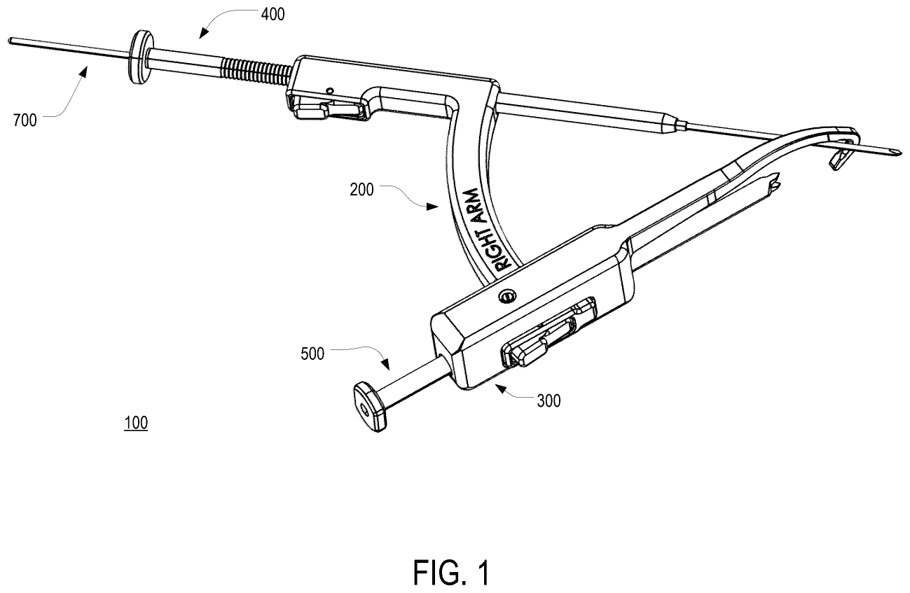

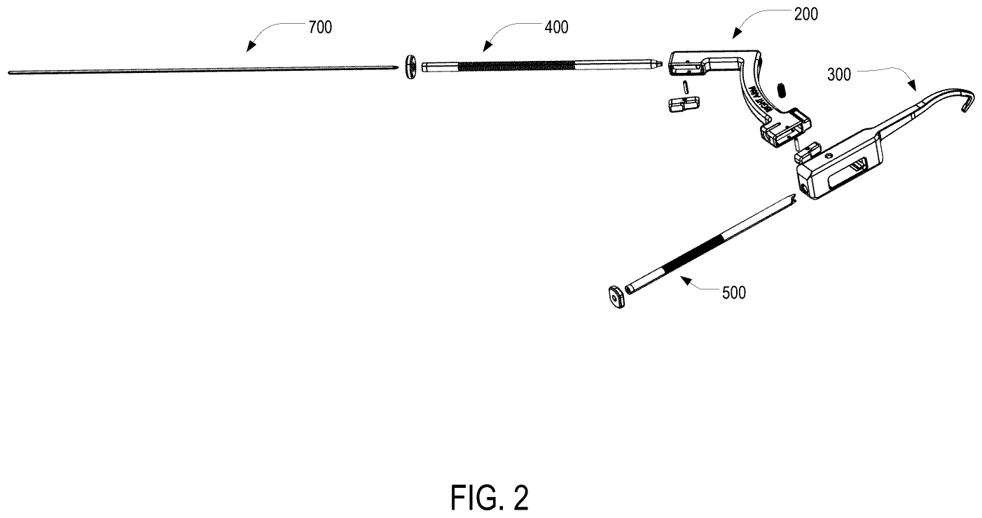

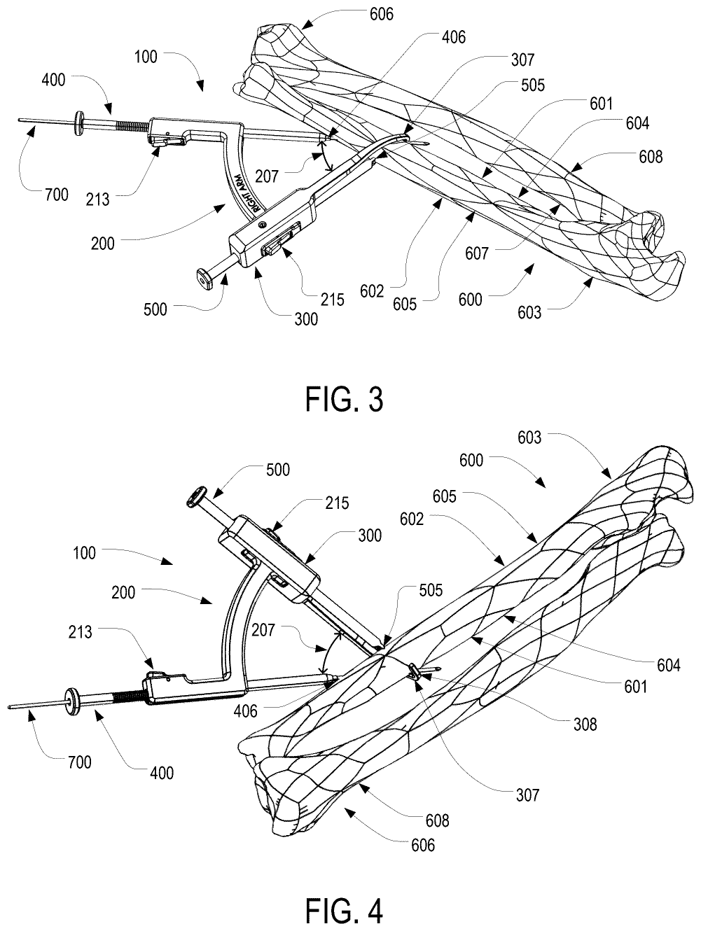

Referring to FIGS. 1, 3 and 4, shown are various isometric views of a guide (100) suitable for drilling a tunnel through a bone (600) at a drill angle (207). The guide comprises a guide arm (200), a guide body (300), a k-wire guide (400), and a locking rod (500) in accordance with one embodiment. Referring to FIG. 2, an exploded view of the guide (100) of FIGS. 1, 3, and 4 is shown. Referring again to FIGS. 3 and 4, the guide (100) is shown engaged with a bone (600) in accordance with one embodiment. The bone further comprises a distal side (601) and a proximal side (602). The bone (600) may optionally be an ulna (603) having a distal side (604) and proximal side (605), or a radius (606) having a distal side (607) and proximal side (608). For illustrative purposes only, the bone (600) with which the guide (100) is shown engaged is an ulna (603) of the forearm. However, the guide (100) is not limited to this use or depiction. For example, the guide (100) is also adapted to engage with a radius (606) of the forearm.

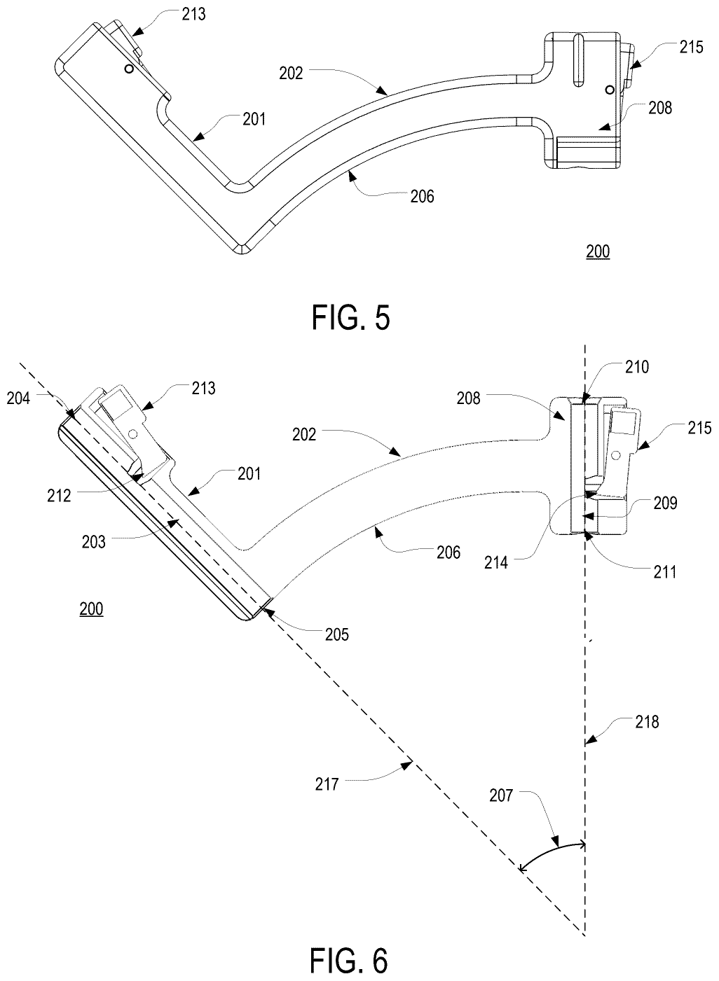

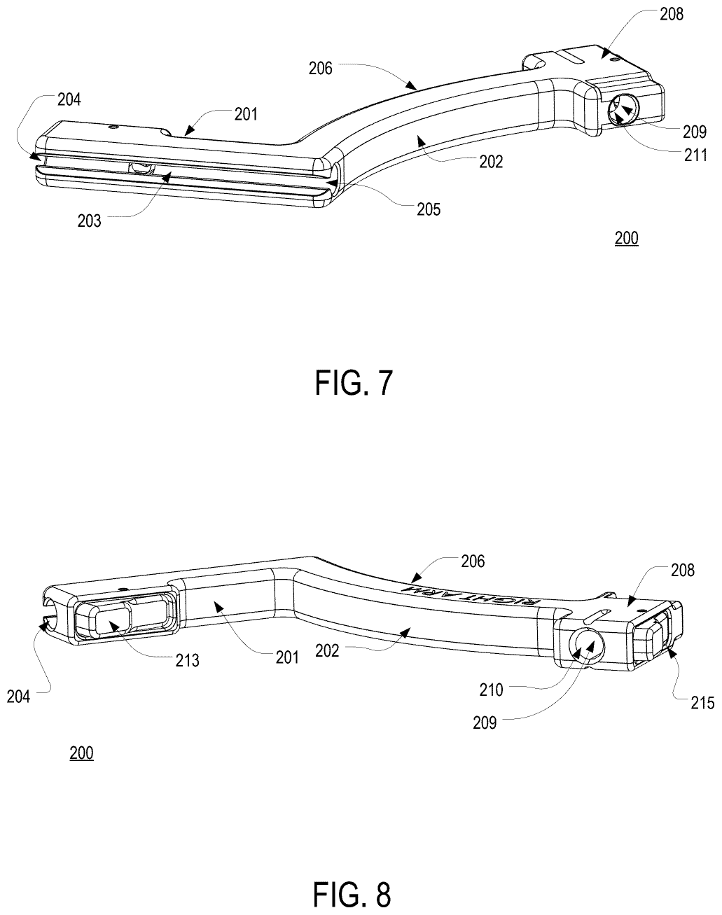

Referring now to FIGS. 5 through 8, a guide arm (200) in accordance with at least one embodiment is shown. The guide arm (200) comprises an elongated segment (201) joined to a connecting segment (202). In at least some embodiments, such as the ones depicted in the figures, the connecting segment (202) may optionally be arcuate. However, the connecting segment (202) may be a number of different shapes and sizes. Referring to FIG. 6, a cross-sectional view of the guide arm (200) is shown. As shown in FIGS. 6 and 7, the elongated segment (201) of the guide arm (200) comprises a k-wire guide tunnel (203) having an entry (204) and an exit (205). The k-wire guide tunnel (203) may further comprise a longitudinal axis (217). Referring further to FIGS. 6 through 8, the connecting segment (202) further comprises a distal end (208) with a guide arm tunnel (209) having a longitudinal axis (218). The guide arm tunnel (209) further comprises an entry (210) and an exit (211). Referring to FIG. 6, in at least some embodiments, the longitudinal axis (218) of the guide arm tunnel (209) and the longitudinal axis (217) of the k-wire guide tunnel (203) converge at an angle equal to the drill angle (207). In embodiments having an arcuate connecting segment (202), the arcuate connecting segment (202) optionally describes an arc (206) equal to the drill angle (207) and the guide arm tunnel (209) is oriented substantially normal to the arc (206).

In at least one embodiment, the drill angle (207) equals 45 degrees. Although the guide is illustrated with a connecting segment (202) having an arc (206) forming a drill angle (207) equal to approximately 45 degrees, this depiction is for illustrative purposes only as various drill angles (207) and various shapes of the connecting segment (202) are within the scope of the present system. In some embodiments, the connecting segment (202) and drill angle (207) may be adjusted to fit different patient anatomies.

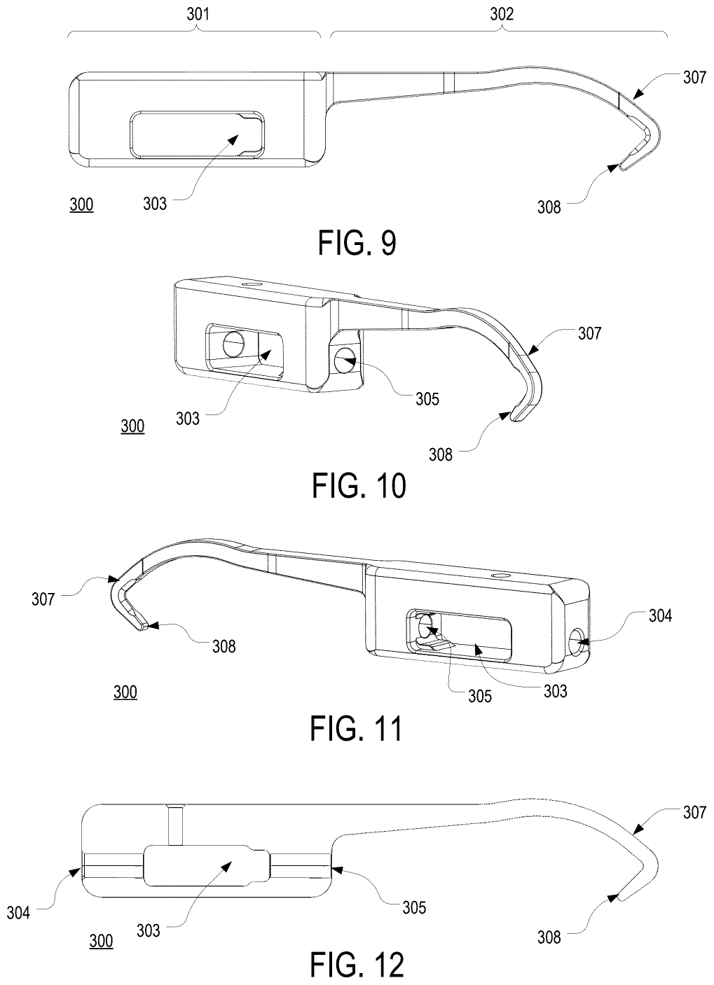

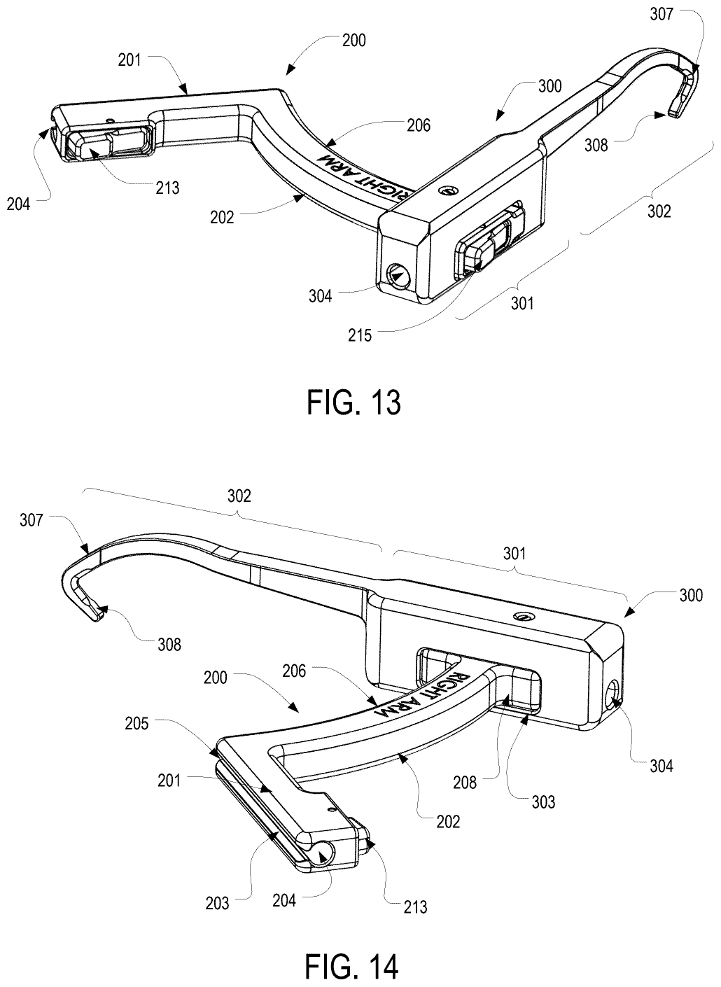

Referring now to FIGS. 9 through 12, shown are various views of a guide body (300) in accordance with one embodiment. The guide body (300) comprises a first section (301) and a second section (302), as shown in FIG. 9. The first section (301) of the guide body (300) comprises an opening (303) adapted to receive the distal end (208) of the guide arm (200) connecting segment (202), as shown in FIGS. 13 through 15. The opening (303) on the first section (301) of the guide body (300) permits the guide body (300) to be separated from the distal end (208) of the guide arm (200) so that the guide body (300) may be inverted, allowing for use of the guide (100) on contralateral sides of the body and for ease of interchangeability during a surgical procedure.

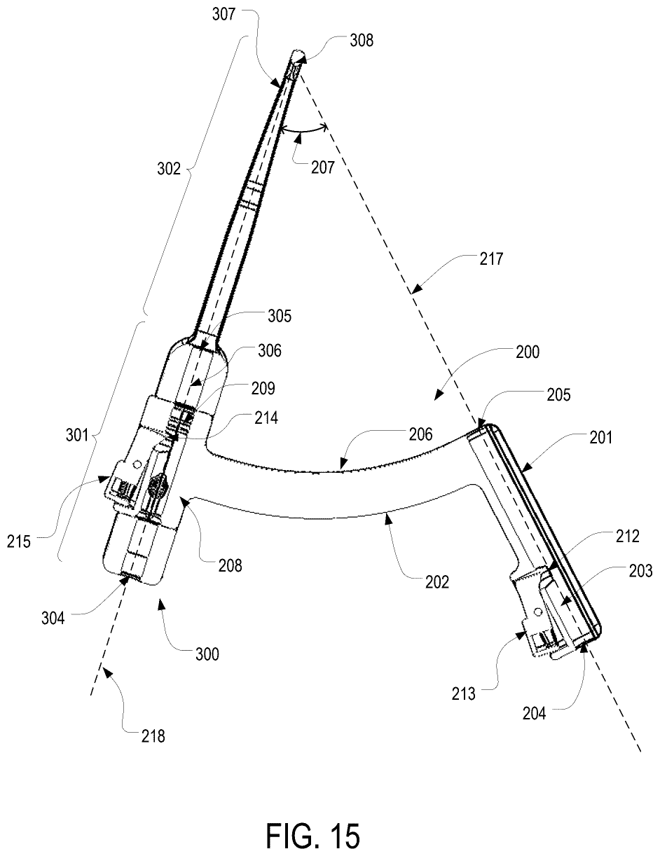

Referring again to FIGS. 9 through 12, the first section (301) of the guide body (300) further comprises an upper locking rod hole (304) and a lower locking rod hole (305). Referring again to FIGS. 13 through 15, shown are various views of the guide body (300) having received the distal end (208) of the guide arm (200) connecting segment (202). Referring to FIG. 15, the upper locking rod hole (304) and the lower locking rod hole (305) of the guide body (300) are adapted to align with the guide arm tunnel (209) to form a locking rod tunnel (306). As further shown in FIG. 15, in at least some embodiments having an arcuate connecting segment (202) forming an arc (206), when the distal end (208) of the guide arm (200) connecting segment (202) is inserted into the opening (303) of the guide body (300), the guide body (300) is oriented substantially normal to the arc (206).

Referring back to FIGS. 9 through 12, the second section (302) of the guide body (300) further comprises a hook (307) terminating in an optional hook tip (308). In at least one embodiment, the hook (307) is adapted to match the curvature of the bone (600). The hook tip (308) is adapted to engage a distal side (601) of the bone (600), as shown in FIG. 4. The hook tip (308) is further adapted to secure the guide body (300) at the ideal trajectory for the drill angle (207). In some embodiments, the hook tip (308) may comprise a sharp point adapted to engage the bone (600). In other embodiments, the hook tip (308) may be a plurality of sharp tips or points, a prong, or a textured surface.

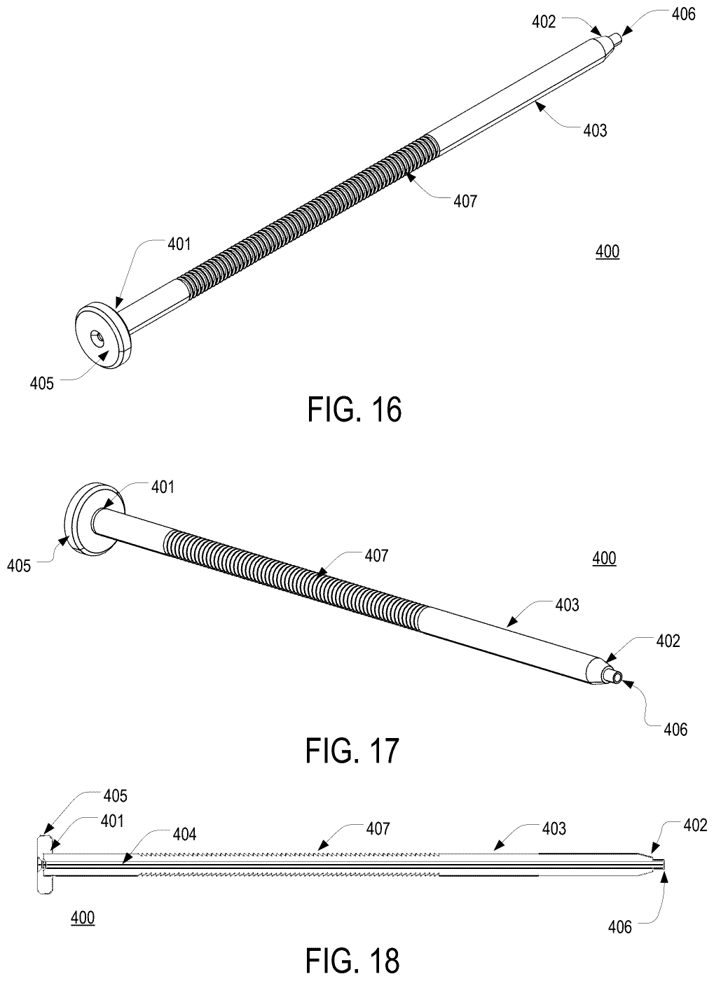

Referring now to FIGS. 16 through 18, shown are various views of a k-wire guide (400) in accordance with one embodiment. The k-wire guide (400) comprises a proximal end (401) and a distal end (402), and a shaft (403) therebetween. In some embodiments, the shaft (403) may optionally be substantially cylindrical. In other embodiments, the shaft (403) may be other shapes. As shown in FIG. 18, the shaft (403) further comprises a k-wire tunnel (404) bored through the shaft (403) of the k-wire guide (400). The k-wire tunnel (404) is adapted to receive a k-wire (700), as shown in FIGS. 1, 3 and 4. In other embodiments, the k-wire tunnel (404) may be adapted to receive other tools. Referring again to FIGS. 16 through 18, the proximal end (401) of the k-wire guide (400) may optionally further comprise a push pad (405), and the distal end (402) of the k-wire guide (400) may optionally further comprise a tip end (406). The tip end (406) is adapted to engage a proximal side (602) of the bone (600), as shown in FIGS. 3 and 4. In at least one embodiment, the tip end (406) is adapted to permit precise insertion of the k-wire (700) from the proximal side (602) through to the distal side (601) of the bone (600) at the drill angle (207), as shown in FIGS. 3 and 4.

Referring again to FIGS. 16 and 17, the shaft (403) of the k-wire guide (400) further comprises a plurality of ratchet teeth (407). The plurality of ratchet teeth (407) is adapted for locking and unlocking the k-wire guide (400) at a desired position in the k-wire guide tunnel (203). In at least some embodiments, the plurality of ratchet teeth (407) may be radially oriented or straight horizontal cuts. Referring back to FIG. 6, the guide arm (200) further comprises a k-wire guide ratchet mechanism (212) adapted to engage the ratchet teeth (407) on the k-wire guide (400) shaft (403). The k-wire guide ratchet mechanism (212) is further adapted to permit the k-wire guide (400) to slide freely in a direction from the entry (204) to the exit (205) of the k-wire guide tunnel (203), and to prevent the k-wire guide (400) from sliding in a direction from the exit (205) to the entry (204) of the k-wire guide tunnel (203).

In a further embodiment, the guide (100) may comprise a k-wire guide button (213), as shown in FIGS. 5, 6, 8, and 13 through 15, to disengage the k-wire guide ratchet mechanism (212) from the ratchet teeth (407) on the k-wire guide (400) and permit the k-wire guide (400) to slide in the direction from the exit (205) to the entry (204) of the k-wire guide tunnel (203).

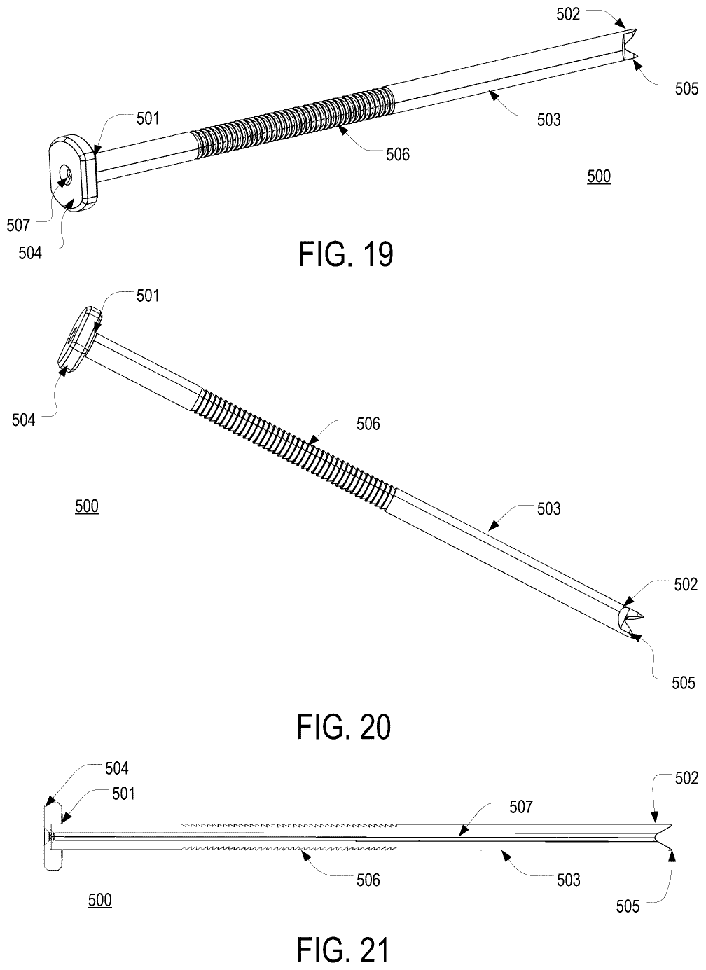

Referring now to FIGS. 19 through 21, shown are various views of a locking rod (500) in accordance with one embodiment. The locking rod (500) comprises a proximal end (501), a distal end (502), and a shaft (503) therebetween. In some embodiments, the shaft (503) may optionally be substantially cylindrical. In other embodiments, the shaft (503) may be other shapes. In at least one embodiment, the locking rod (500) may optionally further comprise a tunnel (507) bored through the shaft (503) of the locking rod (500) (as shown in FIG. 21). The tunnel (507) may be adapted to receive a tool or k-wire. The locking rod (500) is adapted to be received by the locking rod tunnel (306) that is formed by the upper locking rod hole (304) and lower locking rod hole (305) of the guide body (300) aligning with the guide arm tunnel (209) of the guide arm (200), as shown in FIG. 15. Referring to FIGS. 1, 3 and 4, the locking rod (500) is shown received by the guide arm (200) and the guide body (300).

Referring again to FIGS. 19 through 21, the proximal end (501) of the locking rod (500) may optionally further comprise a push pad (504). The distal end (502) of the locking rod (500) further comprises a tip end (505) adapted to engage a proximal side (602) of the bone (600), as shown in FIGS. 3 and 4. In some embodiments, the tip end (505) may be a v-tip end, or comprise a sharp tip or point, a plurality of sharp tips or points, a prong, or a textured surface. The tip end (505) is further adapted to secure the guide body (300) and the guide (100) at the ideal trajectory for the drill angle (207) when the guide arm (200) connecting segment (202) is inserted into the opening (303) of the guide body (300) and the locking rod (500) is inserted into the locking rod tunnel (306).

Referring again to FIGS. 19 through 21, the shaft (503) of the locking rod (500) further comprises a plurality of ratchet teeth (506). The plurality of ratchet teeth (506) is adapted for locking and unlocking the locking rod (500) at a desired position in the locking rod tunnel (306). In at least some embodiments, the plurality of ratchet teeth (506) may be radially oriented or straight horizontal cuts. Referring back to FIG. 6, the guide arm (200) further comprises a locking rod ratchet mechanism (214) adapted to engage the ratchet teeth (506) on the shaft (503) of the locking rod (500). The locking rod ratchet mechanism (214) is further adapted to permit the locking rod (500) to slide freely in a direction from the upper locking rod hole (304) to the lower locking rod hole (305), and to prevent the locking rod (500) from sliding in a direction from the lower locking rod hole (305) to the upper locking rod hole (304).

In a further embodiment, the guide (100) may comprise a locking rod button (215), as shown in FIGS. 5, 6, 8, 13 and 15, to disengage the locking rod ratchet mechanism (214) from the ratchet teeth (506) on the shaft (503) of the locking rod (500) and permit the locking rod (500) to slide in the direction from the lower locking rod hole (305) to the upper locking rod hole (304).

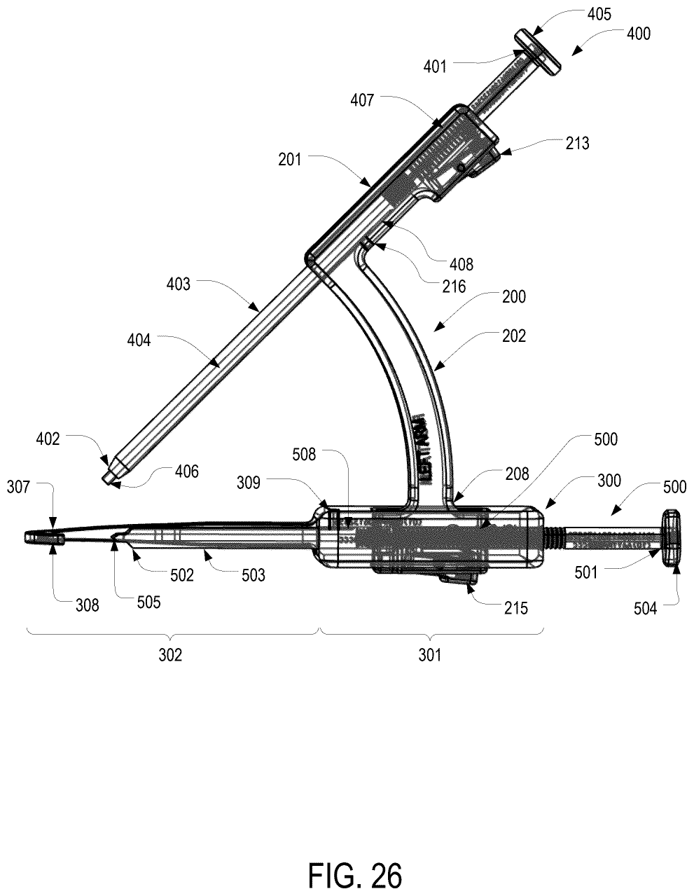

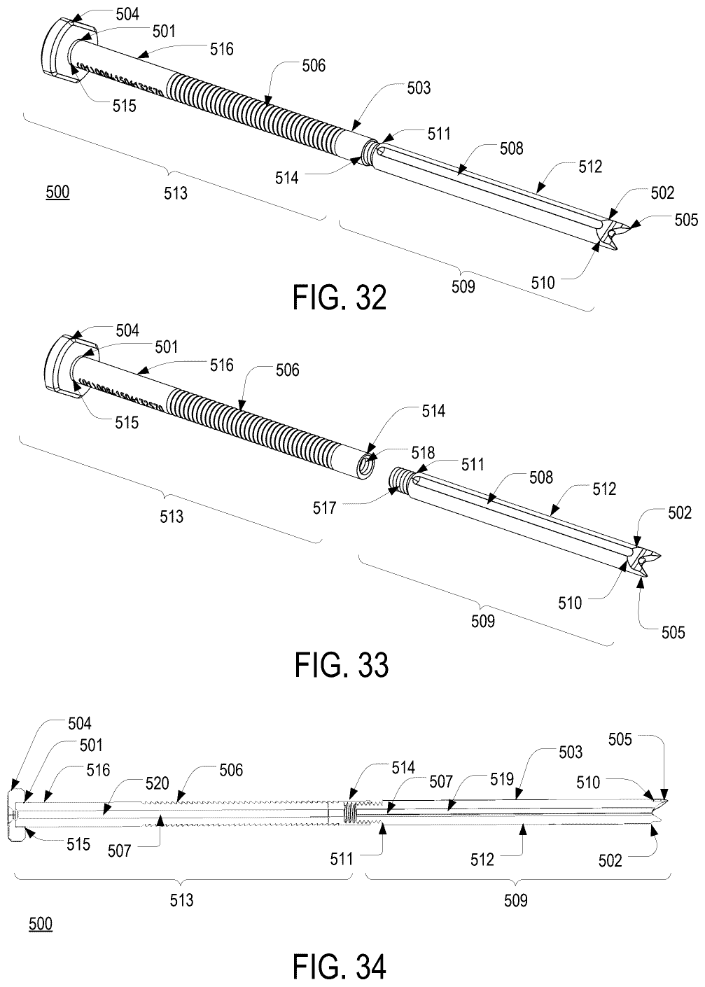

Next, referring to FIG. 26, shown is a bottom view of another embodiment, wherein the guide arm (200), the guide body (300), the k-wire guide (400) and the locking rod (500) are shown, for illustrative purposes only, as transparent to show additional features. FIG. 28, depicts an enlarged view of the guide body (300) of FIG. 26 having received the locking rod (500) in accordance with one embodiment, wherein, for illustrative purposes only, the guide body (300) and the locking rod (500) are depicted as transparent. Referring to both FIGS. 26 and 28, in at least one embodiment, the shaft (503) of the locking rod (500) may optionally further comprise a channel (508) at the distal end (502) of the locking rod (500) (see, for example, FIG. 32), and the first section (301) of the guide body (300) may optionally further comprise a removeable pin (309) that is perpendicular to the axis of the locking rod tunnel (306). The pin (309) of the guide body (300) may be adapted to intersect with the locking rod tunnel (306). The pin (309) of the guide body (300) may be further adapted to engage the channel (508) on the shaft (503) of the locking rod (500) to secure the locking rod (500) when the locking rod (500) is received by the locking rod tunnel (306). The pin (309) of the guide body (300) may be further adapted to prevent rotation of the locking rod (500) when the locking rod (500) is received by the locking rod tunnel (306).

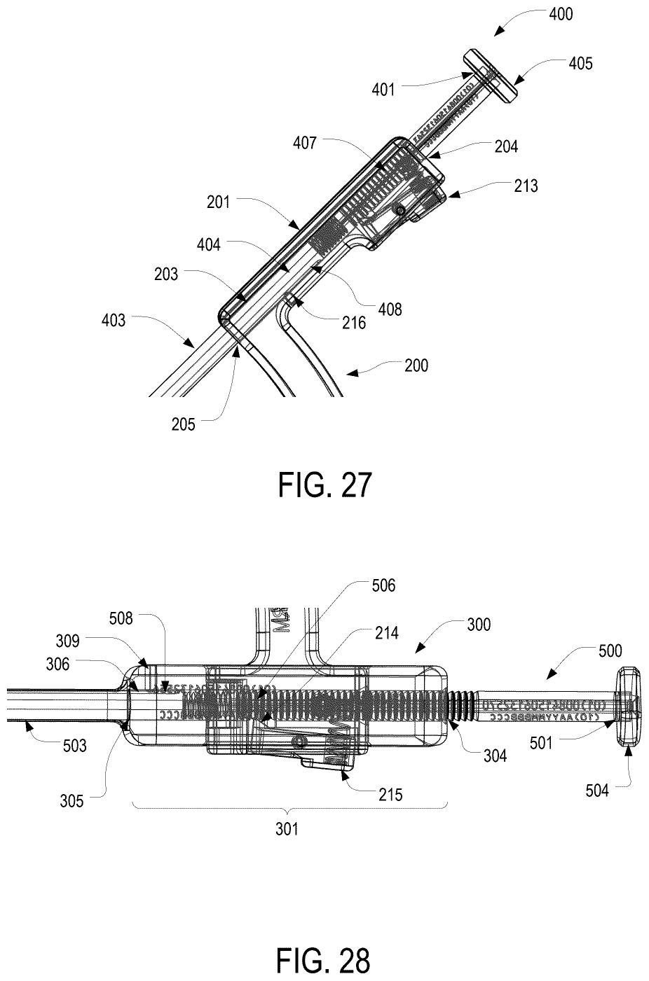

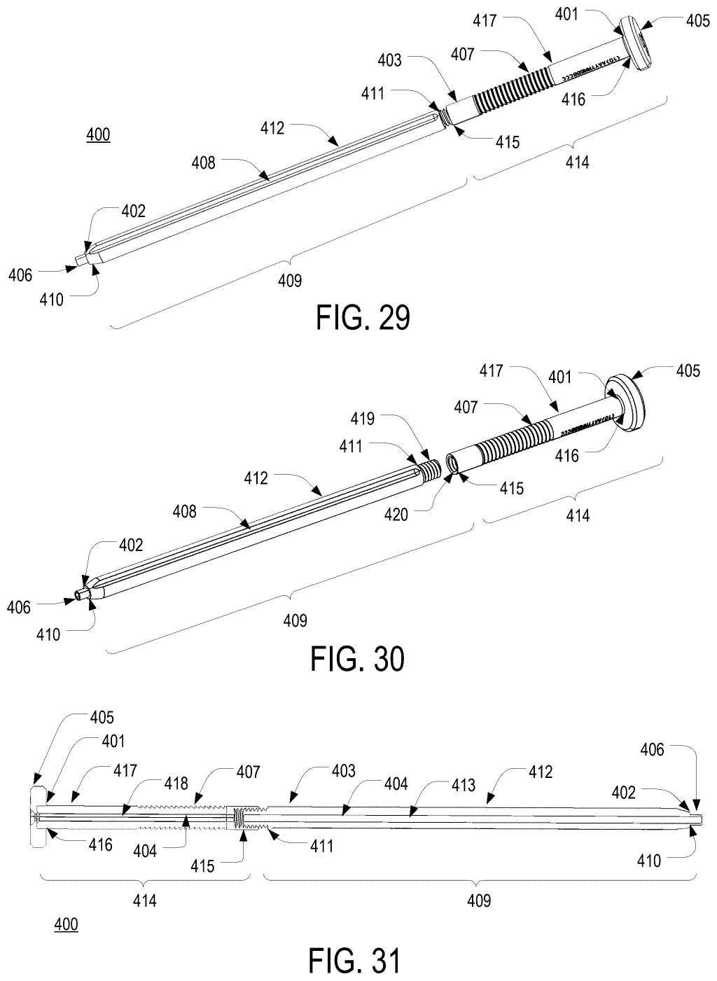

Referring next to FIG. 27, shown is an enlarged view of the elongated segment (201) of the guide arm (200) of FIG. 26 having received the k-wire guide (400) in accordance with one embodiment, wherein, for illustrative purposes only, the guide arm (200) and the k-wire guide (400) are depicted as transparent. Referring to both FIGS. 26 and 27, in at least one other embodiment, the shaft (403) of the k-wire guide (400) may optionally further comprise a channel (408) at the distal end (402) of the k-wire guide (400) (see FIG. 29), and the elongated segment (201) of the guide arm (200) may optionally further comprise a removeable pin (216) that is perpendicular to the axis of the k-wire guide tunnel (203). The pin (216) of the guide arm (200) may be adapted to intersect with the k-wire guide tunnel (203). The pin (216) of the guide arm (200) may be further adapted to engage the channel (408) on the shaft (403) of the k-wire guide (400) to secure the k-wire guide (400) when the k-wire guide (400) is received by the k-wire guide tunnel (203). The pin (216) of the guide arm (200) may be further adapted to prevent rotation of the k-wire guide (400) when the k-wire guide (400) is received by the k-wire guide tunnel (203).

Referring next to FIGS. 29 through 31, in one further embodiment, the k-wire guide (400) may be separated into more than one segment. In one embodiment, such as the one depicted in FIGS. 29 through 31, the k-wire guide (400) may further comprise a distal segment (409) and a proximal segment (414). The distal segment (409) may be located at the distal end (402) of the k-wire guide (400). The distal segment (409) comprises a distal end (410), a proximal end (411), and a shaft (412) therebetween with a distal segment tunnel (413) bored through the shaft (412) (see FIG. 31). The shaft (412) may optionally be substantially cylindrical in some embodiments, or it may other shapes in other embodiments. In embodiments having a distal segment (409), the distal end (410) of the distal segment comprises the optional tip end (406) of the k-wire guide (400), and the shaft (412) of the distal segment (409) may optionally further comprise the channel (408) of the k-wire guide (400) that is adapted to be engaged by the optional pin (216) of the elongated segment (201) of the guide arm (200) (see FIG. 27). The proximal segment (414) may be located at the proximal end (401) of the k-wire guide (400). The proximal segment (414) comprises a distal end (415), a proximal end (416), and a shaft (417) therebetween with a proximal segment tunnel (418) bored through the shaft (417) (see FIG. 31). The shaft (417) may optionally be substantially cylindrical in some embodiments, or it may be other shapes in other embodiments. In embodiments having a proximal segment (414), the proximal end (416) of the proximal segment (414) optionally comprises the push pad (405) of the k-wire guide (400), and the shaft (417) of the proximal segment (414) further comprises the plurality of ratchet teeth (407) of the k-wire guide (400).

In at least some embodiments having a k-wire guide (400) with a proximal segment (414) and a distal segment (409), the optional channel (408) of the k-wire guide (400) and the optional pin (216) of the elongated segment (201) may be adapted to prevent rotation of the k-wire guide (400) distal segment (409) while allowing linear translation of the k-wire guide (400) distal segment (409) and rotation of the k-wire guide (400) proximal segment (414).

The proximal end (411) of the distal segment (409) and the distal end (415) of the proximal segment (414) are adapted to removably attach to form the k-wire guide (400) (see FIG. 29). In some embodiments, such as the one depicted in FIG. 30, the proximal end (411) of the distal segment (409) may optionally have an external helical thread (419) and the distal end (415) of the proximal segment (414) may optionally have an internal helical thread (420) adapted to rotationally engage the external helical thread (419) of the proximal end (411) of the distal segment (409) to secure the distal segment (409) and the proximal segment (414) together and form the k-wire guide (400) (see FIG. 29). In at least one other embodiment (not shown), the distal end (415) of the proximal segment (414) may optionally have an external helical thread and the proximal end (411) of the distal segment (409) may optionally have an internal helical thread adapted to engage the external helical thread of the distal end (415) of the proximal segment (414) to secure the distal segment (409) and the proximal segment (414) together to form the k-wire guide (400). In other embodiments, the proximal end (411) of the distal segment (409) and the distal end (415) of the proximal segment (414) may comprise other attachment mechanisms.

Referring again to FIGS. 29 through 31, when the distal segment (409) and the proximal segment (414) are attached, the shaft (412) of the distal segment (409) and the shaft (417) of the proximal segment (414) (see FIG. 30) are adapted to form the shaft (403) of the k-wire guide (400) (shown attached in FIG. 29), and the distal segment tunnel (413) and the proximal segment tunnel (418) are adapted to align to form the k-wire tunnel (404) (see FIG. 31).

Now referring to FIGS. 32 through 34, in at least one further embodiment, the locking rod (500) may be separated into more than one segment. In one embodiment, the locking rod (500) may further comprise a distal segment (509) and a proximal segment (513). The distal segment (509) may be located at the distal end (502) of the locking rod (500). The distal segment (509) comprises a distal end (510), a proximal end (511), and a shaft (512) therebetween. The shaft (512) may optionally be substantially cylindrical in some embodiments, or it may be other shapes in other embodiments. In embodiments having a distal segment (509), the distal end (510) of the distal segment (509) may comprise the optional tip end (505) of the locking rod (500), and the shaft (512) of the distal segment (509) may optionally further comprise the channel (508) adapted to be engaged by the optional pin (309) of the first section (301) of the guide body (300) (see FIG. 28). The proximal segment (513) may be located at the proximal end (501) of the locking rod (500). The proximal segment (513) comprises a distal end (514), a proximal end (515), and a shaft (516) therebetween. The shaft (516) may optionally be substantially cylindrical in some embodiments, or it may be other shapes in other embodiments. In embodiments having a proximal segment (513), the proximal end (515) of the proximal segment (513) optionally comprises the push pad (504) of the locking rod (500), and the shaft (516) of the proximal segment (513) further comprises the plurality of ratchet teeth (506) of the locking rod (500).

In at least some embodiments having a locking rod (500) with a proximal segment (513) and a distal segment (509), the optional channel (508) of the locking rod (500) and the optional pin (309) of the first section (301) of the guide body (300) may be adapted to prevent rotation of the locking rod (500) distal segment (509) while allowing linear translation of the locking rod (500) distal segment (509) and rotation of the locking rod (500) proximal segment (513).

The proximal end (511) of the distal segment (509) and the distal end (514) of the proximal segment (513) are adapted to removably attach to form the locking rod (500) (see FIG. 32). In some embodiments, such as the one depicted in FIG. 33, the proximal end (511) of the distal segment (509) may optionally have an external helical thread (517) and the distal end (514) of the proximal segment (513) may optionally have an internal helical thread (518) adapted to rotationally engage the external helical thread (517) of the proximal end (511) of the distal segment (509) to secure the distal segment (509) and the proximal segment (513) together and form the locking rod (500) (see FIG. 32). In at least one other embodiment (not shown), the distal end (514) of the proximal segment (513) may optionally have an external helical thread and the proximal end (511) of the distal segment (509) may optionally have an internal helical thread adapted to engage the external helical thread of the distal end (514) of the proximal segment (513) to secure the distal segment (509) and the proximal segment (513) together and form the locking rod (500). In other embodiments, the proximal end (511) of the distal segment (509) and the distal end (514) of the proximal segment (513) may comprise other attachment mechanisms.

Referring again to FIGS. 32 through 34, when attached, the shaft (512) of the distal segment (509) and the shaft (516) of the proximal segment (513) (see FIG. 33) are adapted to form the shaft (503) of the locking rod (500) (shown attached in FIG. 32). Referring to FIG. 34, in at least one embodiment wherein the shaft (503) of the locking rod (500) further comprises a tunnel (507) bored through the shaft (503), the distal segment (509) shaft (512) may further comprise a distal segment tunnel (519) bored through the distal segment (509) shaft (512) and the proximal segment (513) shaft (516) may further comprise a proximal segment tunnel (520) bored through the proximal segment (513) shaft (516). In such embodiments, the distal segment tunnel (519) and the proximal segment tunnel (520) are adapted to align to form the tunnel (507) of the locking rod (500) when the distal segment (509) and the proximal segment (513) are attached.

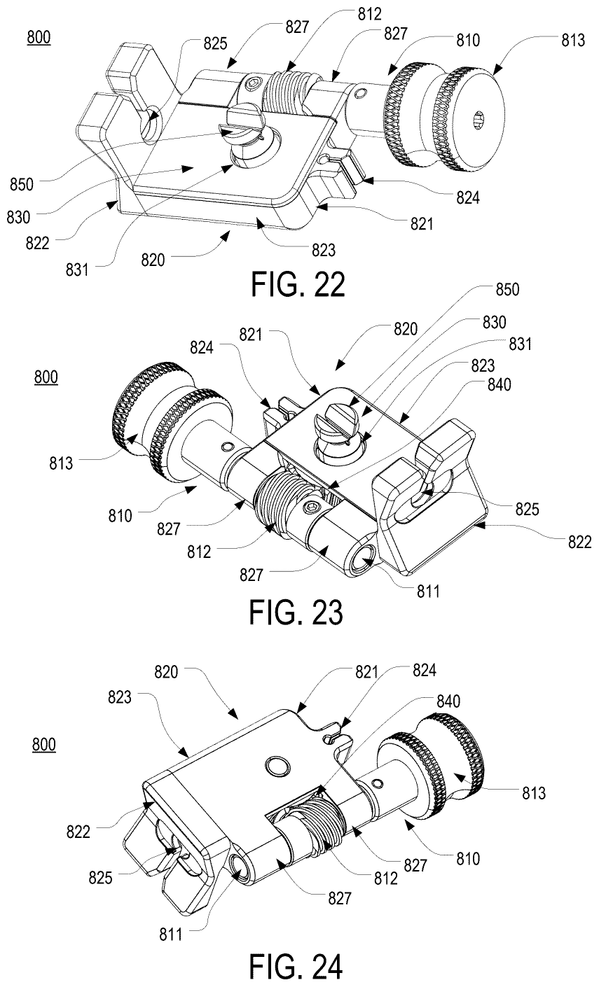

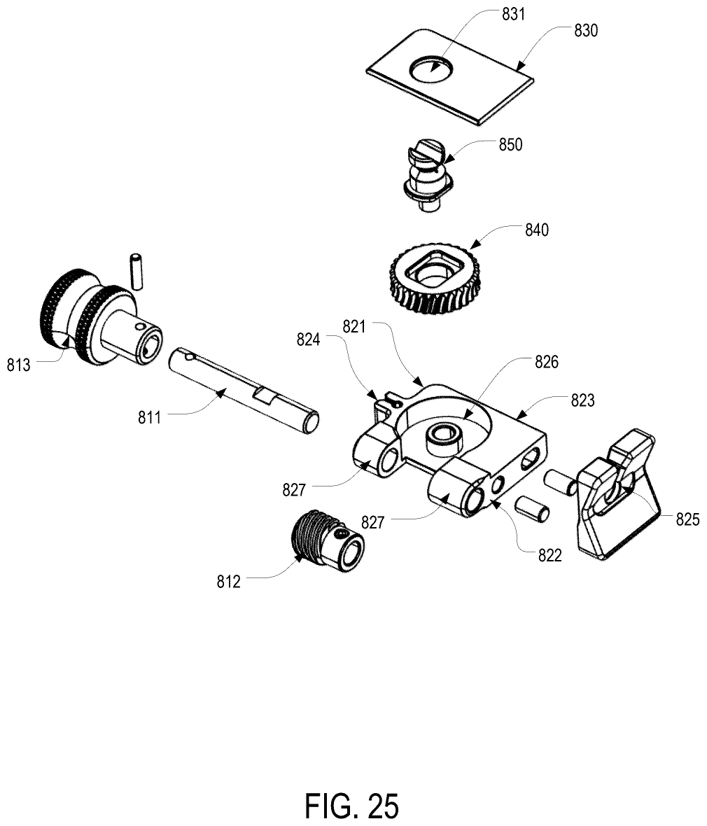

The present system may also comprise a suture tensioner (800) according to the method of use. Referring now to FIGS. 22 through 24, shown are various views of a suture tensioner (800) in accordance with one embodiment. FIG. 25 illustrates an exploded view of the suture tensioner (800) of FIGS. 22 through 24 in accordance with one embodiment. The suture tensioner (800) comprises a knob attachment (810), a base (820), a base cover plate (830), a toothed wheel (840), and a spindle (850).

Referring again to FIGS. 22 through 25, the knob attachment (810) further comprises a shaft (811) that is optionally substantially cylindrical, a worm gear (812), and a thumb knob (813). The thumb knob (813) is adapted to engage and rotate the worm gear (812) when the knob attachment (810), comprising the thumb knob (813), shaft (811) and the worm gear (812), is assembled and the thumb knob (813) is rotated.

Referring again to FIGS. 22 through 25, the base (820) further comprises a proximal end (821), a distal end (822), and a body (823) therebetween. The proximal end (821) further comprises a cleat (824) adapted to hold a suture. The distal end (822) further comprises a recessed hole (825), as shown in FIGS. 23 through 25, adapted to receive a suture button and a suture. Referring to FIG. 25, the body (823) of the base (820) further comprises an opening (826) adapted to receive the toothed wheel (840), and at least two loops (827) adapted to receive the shaft (811) of the knob attachment (810). The worm gear (812) of the knob attachment (810) is adapted to fit between the at least two loops (827) and to permit the shaft (811) of the knob attachment to pass through the at least two loops (827) and the worm gear (812), as shown assembled in FIGS. 22 through 24.

Referring again to FIG. 25, the base cover plate (830) further comprises a hole (831) adapted to receive the spindle (850). The base cover plate (830) is adapted to be received by the body (823) of the base (820), as shown assembled in FIGS. 22 and 23. Referring again to FIG. 25, the opening (826) on the body (823) of the base (820) is adapted to receive the toothed wheel (840) prior to the base cover plate (830) being received by the body (823) of the base (820), as shown assembled in FIGS. 22 and 23. The toothed wheel (840) is adapted to receive the spindle (850).

Referring back to FIGS. 23 and 24, the worm gear (812) of the knob attachment (810) is adapted to engage and rotate the toothed wheel (840) when the toothed wheel (840) is received by the opening (826) on the body (823) of the base (820) and when the worm gear (812) is rotated by the thumb knob (813) on the knob attachment (810). The toothed wheel (840) is then adapted to rotate the spindle (850) when the worm gear (812) is rotated by the thumb knob (813).

In at least one embodiment, the assembled suture tensioner (800) is adapted to tighten an end of a suture when the suture is passed through the spindle (850) and the suture is locked in place using the cleat (824) on the body (823) of the base (820), and when the thumb knob (813) is rotated to rotate the worm gear (812) which engages the toothed wheel (840) that rotates the spindle (850).

### Method of Use

In operation, the following is a listing of the steps typically taken to utilize the guide (100) to drill corresponding angled tunnels through the radius (606) and ulna (603) bones for interosseous ligament (IOL) repair. It should be noted that although the following steps are described in connection with passing the suture and button from the radius (606) to the ulna (603), in at least one embodiment the reverse order of operations, in which the suture and button are passed from the ulna (603) to the radius (606), is also within the scope of the method of use to utilize the guide (100). Further, although the method is described in connection with a radius (606) and ulna (603), the present guide (100) may also be used to facilitate drilling a bone tunnel at an angle through other bones.

1. For superficial exposure for the proximal radial insertion, at the proximal 1/3 of the radius (606), make a 4-6 cm incision on the dorsal and lateral aspect of the forearm using a Thompson Approach. Identify the interval between the Extensor Digitorium Communis and the Extensor Carpi Radialis Brevis (ECRB). Dissect and identify the Pronator Teres (PT) insertion at the radial shaft. The insertion of the central band on the radius (606) is immediately proximal to this structure and on the opposite, sharp or medial edge of the radius (606).

2. For superficial exposure of the distal ulna (603), make an incision centered 6-8 cm proximal to the styloid process over the subcutaneous border of the ulna (603).

3. For deep exposure, identify the Extensor Carpi Ulnaris (ECU) and Flexor Carpi Ulnaris (FCU). Dissect between them and identify the distal origin of the Central Band on the opposite, lateral or sharp edge of the ulna (603). Protect the posterior sensory branch of the Ulnar nerve and the motor branches to Extensor Pollicus Longus (EPL) and Extensor Indicis Proprius (EIP).

4. Locate the Central Band's insertion on the proximal radius (606), which is just proximal to the Pronator Teres insertion. Once the location has been identified, use a marking pen to mark the desired tunnel location on the proximal side (608) of the radius (606). If preferred, the suture and button may be passed from the ulna (603) to the radius (606).

5. When assembling the guide (100), ensure the guide arm (200) is installed on the proximal side of the guide body (300). Refer to the laser markings on the guide arm (200) to ensure the guide arm (200) is inserted into the opening (303) of the first section (301) of the guide body (300) to form the locking rod tunnel (306) (FIGS. 13-15) at the correct orientation required for the left or right arm.

6. Place the hook (307) of the guide body (300) over the desired insertion point on the radius (606) such that the hook tip (308) of the hook (307) engages the distal side (607) of the radius (606) (see FIGS. 3 and 4 for a similar engagement with the ulna (603)).

7. To secure the guide (100) in place at the drill angle (207), insert the locking rod (500) into the locking rod tunnel (306) and optionally push the push pad (504) on the proximal end (501) of the locking rod (500) so that the locking rod (500) tip end (505) engages the proximal side (608) of the radius (606) (see FIGS. 3 and 4 for a similar engagement with the ulna (603)).

8. Insert the k-wire guide (400) through the k-wire guide tunnel (203) of the guide arm (200) elongated segment (201) and optionally push the push pad (405) of the k-wire guide (400) so that the tip end (406) of the k-wire guide (400) distal end (402) engages the proximal side (608) of the radius (606) (see FIGS. 3 and 4 for a similar engagement with the ulna (603)). The k-wire guide (400) should be secured against the surface of the radius (606).

9. Once the guide (100) is fixed in place, insert a 1.4 mm k-wire (700) into the k-wire guide (400) (FIGS. 1, 3 and 4). Drill the k-wire (700) through both cortices of the radius (606) (see the k-wire (700) through the ulna (603) in FIGS. 3 and 4) to prepare the radius (606) for the drilling operation.

10. Remove the guide (100) using the k-wire guide button (213) to disengage the k-wire guide (400) and the locking rod button (215) to disengage the locking rod (500) from the bone (600), leaving the 1.4 mm k-wire (700) in place in the radius (606). Confirm visually that the 1.4 mm k-wire (700) exits the radius (606) at the medial sharp border. The k-wire (700) should be aiming in the direction of the distal ulna (603).

11. Once the 1.4 mm k-wire (700) is fixed in the desired trajectory, use fluoroscopic imaging to verify the proper positioning has been achieved prior to using a 3.5 mm cannulated drill.

12. With the 1.4 mm k-wire (700) fixed in place, use a tissue protector and 3.5 mm cannulated drill to create a radial tunnel. After drilling through both cortices of the radius (606), remove the 1.4 mm k-wire (700).

13. Prepare the ulnar incision for guide (100) positioning and tunnel preparation. If a tendon graft is to be used concomitantly, prepare a separate parallel tunnel for graft placement.

14. For the distal ulna (603) guide (100) positioning, locate the distal insertion of the central band on the ulna (603) for the guide (100) placement.

15. The guide (100) does not need to be reassembled when switching from the radius (606) to the ulna (603) on the same arm. The guide (100) should only be reassembled when used on an opposite arm side (left or right). Place the hook tip (308) of the hook (307) of the guide body (300) at the distal insertion of the central band. The k-wire guide (400) should be aiming in the direction of the proximal radius (606).

16. Once the guide (100) has been fixed in the desired position on the ulna (603), repeat Steps 5 through 11 for drilling operations at the appropriate position on the ulna (603).

17. Through blunt dissection, create a tunnel at the plane of the IOL to pass a suture from the radius (606) to the ulna (603). If preferred, the suture and button may alternatively be passed from the ulna (603) to the radius (606). After creating the ulnar tunnel, use a blunt needle on a toggle hole of an ulnar button on an IOL suture button assembly to pass the ulnar button through the radial and ulnar tunnels.

18. Insert an IOL suture passer into the ulnar incision and through the muscle tunnel into the radial incision. Pass the ulnar button and suture through the loop on the IOL suture passer and pull back, through the intramuscular tunnel previously created, until the ulnar button is retrieved through the ulnar incision. Remove the IOL suture passer once the ulnar button is in position for passage through the ulnar tunnel.

19. Repeat Step 17 on the ulnar side using the toggle needle.

20. After passing the ulnar button through the ulnar tunnel, retrieve the locking suture loops and separate the two loops away from the button holes. While holding the two looped suture ends in place, push the ulnar button towards the bone until it sits flush on the bone surface. The locking suture loops must be held in place by an assistant to prevent suture locking.

21. Once the proper button positioning has been verified, cut the toggle needle suture and remove from the ulnar button hole.

22. Holding the ulnar button against the bone, simultaneously pull on the tightening suture and lock the button against the bone. The locking suture loop can then be released, maintaining compression against the button surface. Care should be taken not to overtighten in acute cases as you may provide excessive negative ulnar variance.

23. Use fluoroscopic imaging to confirm proper radial head height, ulnar variance, and button position.

24. If neutral variance is not reached, retighten the suture by pulling further on the two free tightening suture ends. If it is not possible to restore ulnar variance by hand tensioning, use a suture tensioner as described in steps 26 and 27.

25. After the suture button assembly has been adequately tightened and confirmed fluoroscopically, rotate the forearm in pronation and supination to examine Range of Motion. Full forearm rotation suggests isometric suture construct placement. If required, continue to tighten the suture by repeating the tensioning operations described in steps 26 and 27.

26. If suture tensioning was achieved by hand, skip steps 26 and 27. If use of the suture tensioner (800) is required, place the suture tensioner (800) over the button using the recessed hole (825) at the distal end (822) of the suture tensioner (800) body (823). Once the recessed hole (825) is flush against the button, wrap both tightening suture ends through the spindle (850) and lock the suture in place using the cleat (824) at the proximal end (821) of the suture tensioner (800) body (823).

27. Use the thumb knob (813) of the assembled suture tensioner (800) (FIGS. 22 through 24) to further tension the suture. The thumb knob (813) should be turned clockwise to tighten. Take care not to turn the thumb knob (813) excessively to prevent over tightening. A series of turns should be completed, while evaluating the tensioning in between turns.

28. Once tightened to the desired position remove the suture tensioner (800) by rotating the thumb knob (813) in the opposite direction. Tie a couple half hitches with the tightening sutures if desired then cut near the proximal button.

Although described above in connection with interosseous ligament repair, these descriptions are not intended to be limiting, as other guides can be made in accordance with the description herein, but of different size or scale, for use in other surgical procedures, as desired. As such, although the invention is illustrated and described herein, various modifications and structural changes may be made therein without departing from the spirit of the invention and within the scope and range of equivalents of the claims. Further, any steps described herein may be carried out in any desired order (and any additional steps may be added as desired and/or any steps may be deleted as desired).

Any reference in this specification to “one embodiment,” “an embodiment,” an “example embodiment,” etc., means that a particular feature, structure, or characteristic described in connection with the implementation is included in at least one embodiment of the invention. The appearances of such phrases in various places in the specification are not necessarily referring to the same embodiment. In addition, any elements or limitations of any invention or embodiment thereof disclosed herein can be combined with any and/or all other elements or limitations (individually or in any combination) or any invention or embodiment thereof disclosed herein, and all such combinations are contemplated with the scope of the invention without limitation thereto.

As used herein, the terms “substantial” and “substantially” refer to the complete or nearly complete extent or degrees of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context, which one of ordinary skill in the art would be familiar with. However, generally speaking the nearness of completion will be so as to have the same overall result as if absolute or total completion were obtained.

The use of term “substantial” or “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

Although described above in connection with particular configurations, these descriptions are not intended to be limiting as various modifications may be made therein without departing from the spirit of the invention and within the scope and range of equivalents of the described embodiments. Encompassed embodiments of the present invention can be used in all surgical applications calling for a guide system for drilling an angled tunnel into a bone.

While a number of embodiments of the present invention have been described, it is understood that these embodiments are illustrative only, and not restrictive, and that many modifications may become apparent to those of ordinary skill in the art. For example, any element described herein may be provided in any desired size (e.g., any element described herein may be provided in any desired custom size or any element described herein may be provided in any desired size selected from a “family” of sizes, such as small, medium, large). Further, one or more of the components may be made from many different suitable materials.

In addition, various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A guide for drilling a tunnel through a bone at a drill angle comprising:
a guide arm comprising an elongated segment joined to a connecting segment, the elongated segment comprising a k-wire guide tunnel having a longitudinal axis, an entry, and an exit, the connecting segment comprising a distal end with a guide arm tunnel having a longitudinal axis, the guide arm tunnel having an entry and an exit, the longitudinal axis of the guide arm tunnel and the longitudinal axis of the k-wire guide tunnel converging at an angle equal to the drill angle;
a guide body having a first section and a second section, the first section comprising an opening adapted to receive the distal end of the guide arm connecting segment and having upper and lower locking rod holes, the upper and lower locking rod holes adapted to align with the guide arm tunnel to form a locking rod tunnel, the second section comprising a hook terminating in a hook tip, the hook tip adapted to engage a distal side of the bone;
a k-wire guide adapted to be received by the k-wire guide tunnel, the k-wire guide comprising a proximal end, a distal end, and a shaft, the shaft comprising a k-wire tunnel bored through the shaft, the k-wire tunnel adapted to receive a k-wire, the distal end of the k-wire guide comprising a tip end adapted to engage a proximal side of the bone; the shaft of the k-wire guide comprising a plurality of ratchet teeth adapted for locking and unlocking the k-wire guide at a desired position;
a locking rod adapted to be received by the locking rod tunnel, the locking rod comprising a proximal end, a distal end, and a shaft, the distal end of the locking rod comprising a tip end adapted to engage the proximal side of the bone; the shaft of the locking rod comprising a plurality of ratchet teeth adapted for locking and unlocking the locking rod at a desired position;
the guide arm further comprising a k-wire guide ratchet mechanism adapted to engage the plurality of ratchet teeth on the k-wire guide and to permit the k-wire guide to slide freely in a direction from the entry to the exit of the k-wire guide tunnel, and to prevent the k-wire guide from sliding in a direction from the exit to the entry of the k-wire guide tunnel; and
the guide arm further comprising a locking rod ratchet mechanism adapted to engage the plurality of ratchet teeth on the locking rod and to permit the locking rod to slide freely in a direction from the upper to the lower locking rod hole, and to prevent the locking rod from sliding in a direction from the lower to the upper locking rod hole.

2. The guide of claim 1 further comprising:
a k-wire guide button to disengage the k-wire guide ratchet mechanism from the plurality of ratchet teeth on the k-wire guide and permit the k-wire guide to slide in the direction from the exit to the entry of the k-wire guide tunnel; and
a locking rod button to disengage the locking rod ratchet mechanism from the plurality of ratchet teeth on the locking rod and permit the locking rod to slide in the direction from the lower to the upper locking rod hole.

3. The guide of claim 1 wherein the drill angle is 45 degrees.

4. The guide of claim 1 wherein the connecting segment is arcuate describing an arc equal to the drill angle, and the guide arm tunnel of the distal end is oriented substantially normal to the arc.

5. The guide of claim 1 wherein the plurality of ratchet teeth on the shaft of the k-wire guide are radially oriented.

6. The guide of claim 1 wherein the plurality of ratchet teeth on the shaft of the k-wire guide are straight horizontal cuts.

7. The guide of claim 1 wherein the plurality of ratchet teeth on the shaft of the locking rod are radially oriented.

8. The guide of claim 1 wherein the plurality of ratchet teeth on the shaft of the locking rod are straight horizontal cuts.

9. The guide of claim 1 further comprising:
a channel on the shaft of the locking rod at the distal end of the locking rod;
a pin on the first section of the guide body intersecting with the locking rod tunnel, the pin adapted to engage the channel on the shaft of the locking rod to secure and prevent rotation of the locking rod when the locking rod is received by the locking rod tunnel;
a channel on the shaft of the k-wire guide at the distal end of the k-wire guide; and
a pin on the elongated segment of the guide arm intersecting with the k-wire guide tunnel, the pin adapted to engage the channel on the shaft of the k-wire guide and secure and prevent rotation of the k-wire guide when the k-wire guide is received by the k-wire guide tunnel.

10. The guide of claim 9 wherein the k-wire guide further comprises:
a distal segment at the distal end of the k-wire guide, the distal segment comprising a distal end, a proximal end, and a shaft therebetween with a distal segment tunnel bored through the shaft, the distal end of the distal segment comprising the tip end of the k-wire guide, the shaft of the distal segment further comprising the channel adapted to be engaged by the pin of the elongated segment of the guide arm;
a proximal segment at the proximal end of the k-wire guide, the proximal segment comprising a distal end, a proximal end, and a shaft therebetween with a proximal segment tunnel bored through the shaft, the shaft of the proximal segment further comprising the plurality of ratchet teeth of the k-wire guide;
wherein the proximal end of the distal segment and the distal end of the proximal segment are adapted to removably attach to form the k-wire guide and align the distal segment tunnel with the proximal segment tunnel to form the k-wire tunnel.

11. The guide of claim 10 wherein the distal end of the k-wire guide proximal segment further comprises an internal thread adapted to rotationally engage an external thread of the proximal end of the k-wire guide distal segment.

12. The guide of claim 10 wherein the channel of the k-wire guide and the pin of the elongated segment are adapted to prevent rotation of the k-wire guide distal segment while allowing linear translation of the k-wire guide distal segment and rotation of the k-wire guide proximal segment.

13. The guide of claim 9 wherein the locking rod further comprises:
a distal segment at the distal end of the locking rod, the distal segment comprising a distal end, a proximal end, and a shaft therebetween, the distal end of the distal segment comprising the tip end of the locking rod, the shaft of the distal segment further comprising the channel adapted to be engaged by the pin of the first section of the guide body;
a proximal segment at the proximal end of the locking rod, the proximal segment comprising a distal end, a proximal end, and a shaft therebetween, the shaft of the proximal segment further comprising the plurality of ratchet teeth of the locking rod;
wherein the proximal end of the distal segment and the distal end of the proximal segment are adapted to removably attach to form the locking rod.

14. The guide of claim 13 wherein the distal end of the locking rod proximal segment further comprises an internal thread adapted to rotationally engage an external thread of the proximal end of the locking rod distal segment.

15. The guide of claim 13 wherein the channel of the locking rod and the pin of the first section of the guide body are adapted to prevent rotation of the locking rod distal segment while allowing linear translation of the locking rod distal segment and rotation of the locking rod proximal segment.

16. A method of using a guide to facilitate drilling a tunnel through a bone at a drill angle, the method comprising the steps of:
placing a hook of the guide at a desired insertion point on a distal side of the bone, the guide comprising a k-wire guide tunnel and a locking rod tunnel;
inserting a locking rod into the locking rod tunnel until the locking rod contacts a proximal side of the bone;
inserting a k-wire guide into the k-wire guide tunnel until the k-wire guide contacts the proximal side of the bone such that the k-wire guide forms an angle with the locking rod equal to the drill angle, the k-wire guide comprising a k-wire tunnel;
inserting a k-wire into the k-wire tunnel of the k-wire guide and drilling the k-wire through the proximal side of the bone to the desired insertion point on the distal side of the bone;
removing the k-wire guide from the k-wire guide tunnel of the guide;
removing the locking rod from the locking rod tunnel of the guide;
removing the guide from the bone;
aligning a drill over the k-wire that is inserted in the bone and drilling the drill from the proximal side to distal side of the bone to form a bone tunnel extending from the proximal side to distal side of the bone; and
removing the drill and the k-wire from the bone.

17. The method of claim 16 further comprising repeating the steps at an appropriate position on a second corresponding bone to form a second corresponding bone tunnel at the drill angle.

18. The method of claim 16 further comprising:
engaging a hook tip of the hook with the distal side of the bone when placing the hook of the guide at the desired insertion point on the distal side of the bone; and
disengaging the hook tip of the hook prior to removing the guide from the bone.

19. The method of claim 16 further comprising:
engaging a tip end of the locking rod with the proximal side of the bone when inserting the locking rod into the locking rod tunnel until the locking rod contacts the proximal side of the bone; and
disengaging the tip end of the locking rod prior to removing the locking rod from the locking rod tunnel of the guide.

20. The method of claim 16 further comprising:
engaging a tip end of the k-wire guide with the proximal side of the bone when inserting the k-wire guide into the k-wire guide tunnel until the k-wire guide contacts the proximal side of the bone; and
disengaging the tip end of the k-wire guide prior to removing the k-wire guide from the k-wire guide tunnel of the guide.

21. The method of claim 16 wherein inserting the locking rod into the locking rod tunnel further comprises engaging one of a plurality of ratchet teeth on a shaft of the locking rod with a locking rod ratchet mechanism on the guide.

22. The method of claim 21 further comprising disengaging the locking rod from the guide by pushing on a locking rod button on the guide to release the locking rod ratchet mechanism prior to removing the locking rod from the locking rod tunnel.

23. The method of claim 16 wherein inserting the k-wire guide into the k-wire guide tunnel further comprises engaging one of a plurality of ratchet teeth on a shaft of the k-wire guide with a k-wire guide ratchet mechanism on the guide.

24. The method of claim 23 further comprising disengaging the k-wire guide from the guide by pushing on a k-wire guide button on the guide to release the k-wire guide ratchet mechanism prior to removing the k-wire guide from the k-wire guide tunnel.
